(19) United States
(12) Patent Application Publication
(10) Pub. No.: US 2023/0380462 A1
(43) Pub. Date: Nov. 30, 2023

(54) FERMENTED COMPOSITION

(71) Applicant: MANDA FERMENTATION CO., LTD., Hiroshima (JP)

(72) Inventors: SHINSUKE KISHIDA, Hiroshima (JP); KOTARO FUJIOKA, Hiroshima (JP); ATSUSHI MASUDA, Hiroshima (JP); SAE KORO, Hiroshima (JP); HUANG CHUN-MING, San Diego, CA (US)

(73) Assignee: MANDA FERMENTATION CO., LTD., Hiroshima (JP)

(21) Appl. No.: 18/032,350

(22) PCT Filed: Oct. 20, 2021
(86) PCT No.: PCT/JP2021/038832

§ 371 Date: Apr. 18, 2023

(30) Foreign Application Priority Data
Oct. 20, 2020 (JP) 2020-176185

(51) Int. Cl. A23L 19/00; A61K 35/02; A61K 31/015; A61K 31/07; A61K 31/51; A61K 31/525; A61K 31/4415; A61K 31/355; A61K 31/455; A61K 33/06; A61K 33/42; A61K 33/00; A61K 33/34; A61K 31/198; A61K 31/405; A61K 31/4172; A61K 31/401; A23L 33/135

(52) U.S. Cl. CPC A23L 19/00; A61K 35/02; A61K 31/015; A61K 31/07; A61K 31/51; A61K 31/525; A61K 31/4415; A61K 31/355; A61K 31/455; A61K 33/06; A61K 33/42; A61K 33/00; A61K 33/34; A61K 31/198; A61K 31/405; A61K 31/4172; A61K 31/401; A23L 33/135

(57) ABSTRACT

A prebiotic food product which increases a short-chain fatty acid in an intestine is provided, where the prebiotic food product mainly contains a fermented composition obtained by fermenting and maturing a plurality of ingredients including fruits, citrus, root vegetables such as a burdock and a carrot, cereals, sesames, seaweeds and saccharoses.

### CROSS REFERENCE TO THE RELATED APPLICATIONS

This application is the national phase entry of International Application No. PCT/JP2021/038832, filed on Oct. 20, 2021, which is based upon and claims priority to Japanese Patent Application No. 2020-176185(JP), filed on Oct. 20, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBTBM016_Sequence_Listing.txt, created on 04/06/2023, and is 1,226 bytes in size.

### TECHNICAL FIELD

The present invention relates to a fermented composition that increases the amounts of intestinal bacteria producing a short-chain fatty acid in the intestine, and a short-chain fatty acid.

### BACKGROUND

A short-chain fatty acid is a type of an acid (organic acid) produced by intestinal bacteria in the human large intestine, and specific types thereof include acetic acid, propionic acid, and butyric acid.

In recent years, benefits of short-chain fatty acids have attracted attention. the intestinal mucosa serves an intestinal barrier function to prevent pathogen entry while the intestine is at risk of infection and inflammation caused by a virus or a pathogen that is taken in along with a meal. Butyric acid and propionic acid fortify the intestinal barrier function by maintaining the intestinal mucosa.

Butyric acid is the most important energy source for intestinal epithelial cells and has been reported to promote the intestinal epithelium metabolism and the peristaltic movement of the intestinal tract. In addition, butyric acid has anti-obesity and antidiabetic effects. There is also a report that butyric acid restores an immune response that eliminates an influenza virus, thus being useful for the immune system as well.

These highly useful short-chain fatty acids are difficult to be taken in by eating or drinking due to the odor, taste, and low absorbability. It is effective to allow intestinal bacteria naturally present in the intestine to produce the short-chain fatty acids.

### SUMMARY OF INVENTION

### Technical Problem

The present invention intends to provide a food product that allows intestinal bacteria that innately present in the intestine to produce short-chain fatty acids, and does not intend to directly ingest short-chain fatty acids as a supplement food.

Through repeated tests and experiments, The inventors found that the intestinal bacteria that produce short-chain fatty acids increase in the human intestine when the fermented composition of the present invention is ingested.

### Solution to Problem

The present invention has solved the technical problem using the following technical means.

The present invention provides a prebiotic food product, which main ingredient is a fermented composition featured to increase specific short-chain fatty acids in the intestine, and the fermented composition is obtained by fermentation and maturation of one or more kinds of fruits selected from an apple, a persimmon, a banana, a pineapple, an akebia, silver vine, a fig, a wild strawberry, a strawberry, crimson glory vine, a grape, a mountain peach, a peach, a Japanese apricot, a blueberry, and a raspberry; one or more citrus fruits selected from a navel orange, a hassaku orange, a mandarin orange, a summer tangerine, an orange, an iyokan, a kumquat, a yuzu, a kabosu, a pomelo, a ponkan, a lemon, and a lime; one or more root vegetables selected from a burdock, a carrot, a garlic, a lotus root, and a lily bulb, one or more cereals selected from brown rice, glutinous rice, white rice, millet, corn, wheat, barley, foxtail millet, and Japanese barnyard millet; one or more beans and sesames selected from a soybean, a black bean, black sesame, white sesame, an adzuki bean, and a walnut; one or more seaweeds selected from kelp, wakame seaweed, hijiki seaweed, green laver, and laver; one or more saccharoses selected from brown sugar, fructose, and glucose; and one or more selected from honey, starch, a cucumber, perilla, and celery, and the main component consists of the following constituents and amino acid composition that contains the following per 100 g of main components:
moisture: 5.0 g to 50.0 g,
protein: 0.5 g to 10.0 g,
fat: 0.05 g to 10.00 g,
carbohydrate (sugar): 30.0 g to 75.0 g,
carbohydrate (fiber): 0.1 g to 5.0 g,
ash: 0.5 g to 5.0 g,
β-carotene: 10 μg to 150 μg,
vitamin A effect: 10 IU to 100 IU,
vitamin B1: 0.01 mg to 0.50 mg,
vitamin B2: 0.01 mg to 0.50 mg,
vitamin B6: 0.01 mg to 0.50 mg,
vitamin E: 10.0 mg or less,
niacin: 0.1 mg to 6.0 mg,
calcium: 50 mg to 900 mg,
phosphorus: 200 mg or less,
iron: 1.0 mg to 5.0 mg,
sodium: 20 mg to 300 mg,
potassium: 300 mg to 1000 mg,
magnesium: 40 mg to 200 mg,
sodium chloride equivalent: 0.05 g to 1.00 g, and
copper: 7.0 ppm or less, and
as for the amino acid composition, in 100 g,
isoleucine: 30 to 200 mg,
leucine: 50 to 400 mg,
lysine: 20 to 200 mg,
methionine: 10 to 150 mg,
cystine: 10 to 100 mg,
phenylalanine: 30 to 250 mg,
tyrosine: 20 to 200 mg,
threonine: 40 to 200 mg,
tryptophan: 1 to 100 mg,
valine: 30 to 300 mg,
histidine: 10 to 200 mg,
arginine: 40 to 400 mg,
alanine: 50 to 300 mg,
aspartic acid: 100 to 600 mg,
glutamic acid: 100 to 1200 mg,
glycine: 30 to 300 mg,
proline: 40 to 400 mg, and
serine: 30 to 300 mg,

The present invention provides a prebiotic food product that mainly contains a fermented composition obtained by performing fermentation and maturation of the above-mentioned fermented composition by adding one or more among a mulberry, a ginger or a loquat to the ingredients, consists of the above-mentioned constituents and amino-acid composition.

The prebiotic food product may mainly contain the fermented composition that can increase intestinal bacteria that boost specific short-chain fatty acids in the intestine.

The intestinal bacteria may be Bifidobacterium longum.

The specific short-chain fatty acid may be one or more short-chain fatty acids selected from acetate, butyrate, isobutyrate, isopentanoate, and propionate.

The present invention provides the prebiotic food product that contains the fermented composition as the main ingredient and can increase the production of one or more short-chain fatty acids selected from acetate, propionate, butyrate, and isobutyrate by increasing Lactobacillus acidophilus.

The present invention provides a method to increase the number of intestinal bacteria Bifidobacterium longum in an intestine and increasing one or more short-chain fatty acids selected from acetate, butyrate, isobutyrate, isopentanoate, and propionate when the fermented composition is ingested.

The present invention provides a method to increase intestinal bacteria Lactobacillus acidophilus and promote the production of one or more short-chain fatty acids selected from acetate, propionate, butyrate, and isobutyrate, when the fermented composition is ingested.

### Advantageous Effects of Invention

The present fermented composition enhances the abundance ratio of intestinal bacteria that produce a short-chain fatty acid beneficial to health and increase the short-chain fatty acids, including acetate, butyrate, isobutyrate, isopentanoate, in the internal environment of the large intestine.

Hereinafter, the present invention will be further described by combining the attached drawings and Examples.

### BRIEF DESCRIPTION OF THE DRAWINGS

FIGS. 1A-1B: FIG. 1A is a photograph that shows anaerobic culture of intestinal bacteria in a culture system that uses phenol red as an indicator. FIG. 1B, a graph showing the measurement results of anaerobic culture media of intestinal bacteria in a culture system using phenol red as an indicator at OD₅₆₂ nm.

FIG. 2: Graph showing changes in the abundance ratio of different intestinal bacteria.

FIG. 3: Table showing changes in amounts of different short-chain fatty acids.

FIGS. 4A-4D: Graphs showing concentrations of different short-chain fatty acids produced by Lactobacillus acidophilus.

### DETAILED DESCRIPTION OF THE EMBODIMENTS

The inventors tested whether the fermented composition affects the production of the short-chain acids in the human intestine (in particular, the large intestine).

The present fermented composition in the present embodiment is obtained by performing static fermentation and maturation of brown sugar, fructose, glucose, an apple, a persimmon, a banana, a pineapple, white rice, brown rice, glutinous rice, foxtail millet, barley, millet, corn, a mandarin orange, a hassaku orange, a navel orange, an iyokan, a lemon, a summer tangerine, a kabosu, a kumquat, a pomelo, a ponkan, a yuzu, a grape, an akebia, a fig, silver vine, crimson glory vine, a mountain peach, a strawberry, a Japanese apricot, a soybean, black sesame, white sesame, a black bean, a carrot, a garlic, a burdock, a lily bulb, a lotus root, hijiki seaweed, wakame seaweed, laver, green laver, kelp, honey, a walnut, starch, a cucumber, celery, and perilla.

Bacteria isolated from human stool (10⁵ CFU/ml) were cultured in a reinforced clostridial medium (RCM, Oxford, Hampshire, UK) under anaerobic conditions at 37° C. using the Gas-Pak method (BD, Sparks, MD, USA). The microbial culture was diluted to 1:100, and the diluted microbial culture was cultured until absorbance at 600 nm [optical density (OD) 600] reached 1.0. Microorganisms were obtained by centrifugation at 5,000 g for 10 minutes, and the obtained microorganisms were washed with PBS and suspended in PBS.

Next, the microorganisms obtained above were grown in a eutrophic medium containing brown sugar and the present fermented composition (2%) under an anaerobic condition at 37° C. The microorganisms were anaerobically cultured in a culture system using phenol red as a color indicator. 0.001% (w/v) phenol red (MilliporeSigma) in the rich medium was used as the indicator, which changes the color from red-orange to yellow when fermentation occurs. Fermentation was quantitatively measured at OD₅₆₂ nm in a control liquid medium and a liquid medium that had been cultured for 18 hours.

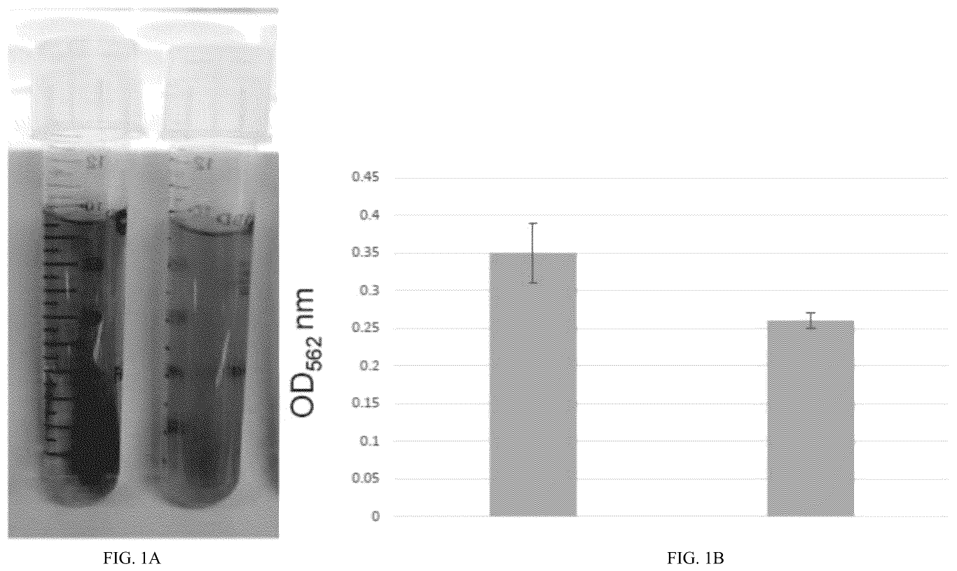

FIG. 1A is a photograph showing an aspect of the intestinal bacteria anaerobically cultured in the culture system using phenol red as an indicator. Change in color from red-orange to yellow is observed when compared with a liquid medium cultured for 18 hours. This indicates that fermentation progressed in the liquid medium. FIG. 1B is a graph showing the measurement results of the control liquid medium and the liquid medium cultured for 18 hours at OD₅₆₂ nm, respectively. The quantitative measurement result indicates 0.25 in the liquid medium cultured for 18 hours, while the result is 0.35 in the control liquid medium.

Next, bacteria were isolated to identify the microorganism (bacteria) that contributed to the fermentation. The test procedure is as follows. A mixture containing trypticase agar medium+(weight/volume) phenol red (manufactured by MilliporeSigma)+2% present fermented composition was smeared on a flat surface, and 100 microliters of stool of a 35-year-old Caucasian male diluted and dispersed in PBS buffer was cultured for 24 hours.

The isolated bacteria from the colony were genetically identified as Bifidobacterium longum. A next-generation sequencing confirmed that the growth of Bifidobacterium was predominant over other bacteria among the plurality of types of bacteria when the stool was cultured using the present fermented composition as a source for assimilation.

The specific microorganism identification method is as follows. DNA or a DNA extract was extracted from a co-culture of brown sugar or the present fermented composition and the stool using QIAamp DNA Stool Mini Kit (QIAGEN GmbH, Hilden, Germany) by an ordinary method according to the instructions of the kit. A 16S rRNA gene analysis method was used for the identification of bacteria. Single colonies were picked up using sterilized toothpick and underwent 16S rRNA gene analysis using a PCR primer pair 27F-534R, and the analysis results were identified using the Basic Local Alignment Search Tool (BLAST) homology search program.

Next, a flora analysis method according to the present invention based on DNA extraction from bacteria and next-generation sequencing will be described. Total DNA was extracted from human stool incubated with brown sugar or the present fermented composition using QIAamp DNA Stool Mini Kit (QIAGEN GmbH, Hilden, Germany) based on the manufacturer instructions. Among extremely high reproducible kits, the Qiagen DNA extraction method was considered to have a high accuracy with minimal impact on next-generation sequence data analysis.

Five samples from each experimental group underwent next-generation sequencing (total number of samples=10). A eubacteria primer kit (Qiagen Inc., Valencia, CA) was used under the following conditions: 94° C. for 3 minutes and then 94° C. for 30 seconds; 28 cycles of 53° C. for 40 seconds and 72° C. for 1 minute, followed by a final extension process at 72° C. for 5 minutes. After the PCR, all amplification products from the different samples were mixed at equal concentrations and purified using Agencourt Ampure beads (Agencourt Bioscience Corporation, MA, USA). Sequencing of the samples was conducted using the Roche 454 FLX titanium apparatus and reagents according to the manufacturer guideline. 16S rRNA gene V4 variable region PCR primer 515/806 was used under the following conditions: 28 cycles (5 cycles for the PCR product) of 94° C. for 3 minutes, then 94° C. for 30 seconds, 53° C. for 40 seconds and 72° C. for 1 minute, followed by a final extension process at 72° C. for 5 minutes; and one PCR for 30 cycles using the HotStarTaq Plus Master Mix Kit (Qiagen, US) were performed.

The sequencing was performed with MR DNA (www.mrdnalab.com, Shallowater, TX, USA) on the Ion Torrent PGM machine according to the manufacturer guideline. Sequence data was processed using a proprietary analysis pipeline (MR DNA, Shallowater, TX, USA). After removing the barcodes and primers from the sequences, a sequence having a length of less than 150 bp was removed, as well as a sequence containing an ambiguous base call and a homopolymer strand having a length of greater than 6 bp. Noise was removed from the sequences, 16S rRNA operational taxonomic units (OTUs) were created, and chimeras were removed. The OTUs were defined by clustering at 3% dissimilarity (97% similarity). The final OTUs were taxonomically classified using BLASTn for a curated database derived from GreenGenes, RDPII, and NCBI (www.ncbi.nlm.nih.gov, http://rdp.cme.msu.edu).

The DNA extraction will be described. A lysate containing DNA is prepared by heating the sample to 100° C. for 10 minutes in a 1× lysis buffer, cooling the heated sample, and performing centrifugation at 18,000×g for 5 minutes to obtain a supernatant. A 10× lysis buffer contains 10% Triton X-100, 5% Tween 20, 100 mM Tris-HCl (pH 8.0), and 10 mM EDTA (Reischl, Linde, Metz, Leppmeier, & Lehn, 2000 PMID: 10835024).

Next, the PCR of 16s rDNA will be described. The conditions of PCR per cycle are set to for 5 minutes and then 15 cycles (first PCR) or 20 cycles (second PCR) of 94° C. for 30 seconds, 54° C. for 40 seconds, 72° C. for 1 minute, and 72° C. for 10 minutes. The lysate (2-10 μl) is used together with AmpliTaq™ gold mix (Thermo Fisher Scientific Inc., Grand Island, NY) in a final volume of 50 μl. The primers for the first PCR are universal 16s bacterial primers (Nakatsuji, et al., 2013 PMID: 23385576) U1048F (5′-GTG-STG-CAY-GGY-TGT-CGT-CA-3′, as shown in SEQ ID NO: 1) and U1175R (5′-ACG-TCR-TCC-MCA-CCT-TCC-TC-3′, as shown in SEQ ID NO: 2). The second PCR is performed using forward adapter primers with barcodes IT-A-BC#-U1048-F (5′-CCA-TCT-CAT-CCC-TGC-GTG-TCT-CCG-ACT-CAG-BC#-GAT-GTG-STG-CAY-GGY-TGT-CGT-CA-3′, as shown in SEQ ID NO: 3, containing the Ion Torrent adapter A sequence and Ion Torrent barcode+“stuffer”, with a single adapter primer containing the Ion Torrent P1b sequence) and P1-U1175-R (5′-CCA-CTA-CGC-CTC-CGC-TTT-CCT-CTC-TAT-GGG-CAG-TCG-GTG-ATA-CGT-CRT-CCM-CAC-CTT-CCT-C-3′, as shown in SEQ ID NO: 4). The final PCR (7 cycles) is performed using KAPA library primers (Kapa Biosystems, Inc., Wilmington, MA) according to the protocol for the kit.

Next, the next-generation sequencing (NGS) of 16s rDNA will be described. Individual sample PCRs are mixed at equal concentrations based on Qubit results, templates are prepared with the OneTouch 2 machine based on the Ion PGM Hi-Q View OT2 kit using the standard protocol, and the sequences are determined with the Ion Torrent PGM machine based on the Ion PGM Hi-Q View sequencing kit for 700 runs using the standard protocol for the Ion 314 V2 chip (all Thermo Fisher Scientific Inc., Grand Island, NY).

The analysis of the results of the next-generation sequencing of 16s rDNA will be described. The 16s rDNA sequences are automatically binned into sample fastq files by the Ion Server software based on the adapter primer barcodes. The primer sequences of the reads are then trimmed, and the quality is screened using a custom python script based on the 10 bp sliding window quality factor being 20 or greater. Next, taxonomy is assigned to the filtered sequences using RDP Classifier v2.12 (Wang, Garrity, Tiedje, & Cole, 2007 PMID: 17586664) in a confidence interval of 80% or greater. The taxonomic profiles are compared using custom shell scripting and QIIME 1.9.1 (Caporaso, et al., 2010 PMID: 20383131).

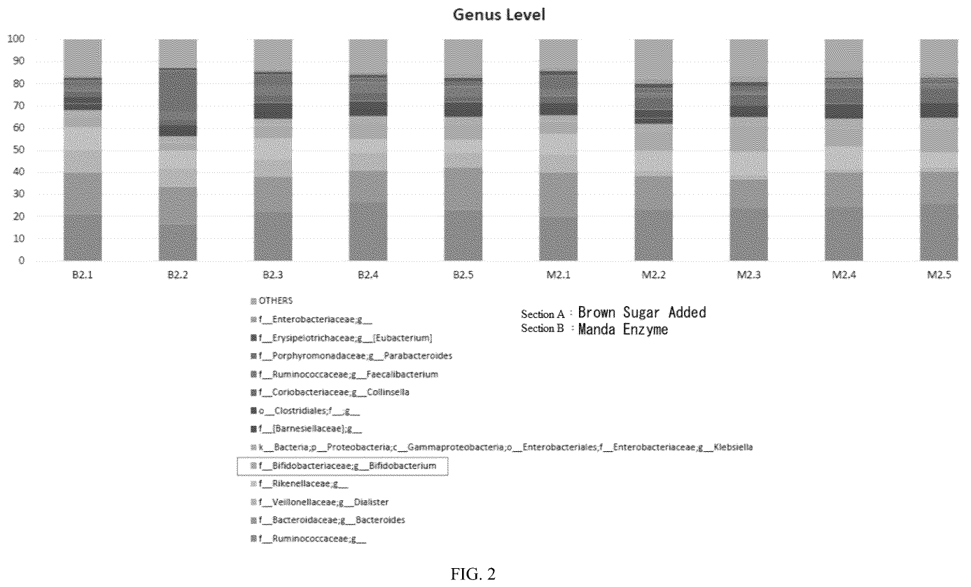

FIG. 2 is a graph showing changes in abundance ratio of various intestinal bacteria. Under the same experimental conditions as above, bacteria isolated from human stool (10⁵ CFU/ml) were cultured for 18 hours in a reinforced clostridial medium (RCM, Oxford, Hampshire, UK) under an anaerobic condition at 37° C. using the Gas-Pak method (BD, Sparks, MD, USA). In the experiment, 5 cases were prepared for each of a control group in which the medium was administered with brown sugar and an experimental group in which the medium was administered with the present fermented composition. The microbial flora analysis method is as described above. The results revealed that the abundance ratio of Bifidobacterium longum increased in the group administered with the present fermented composition in all experimental cases.

Next, it was tested whether short-chain fatty acids increased in the group to which the present fermented composition was administered. Stool isolates (10⁵ CFU/ml) were incubated for 18 hours in a rich medium containing 2% present fermented composition, and the fermentation medium was centrifuged at 5,000 g for 10 minutes to remove bacteria. The pellets of bacteria in the group administered with the present fermented composition are resuspended in 450 μl of a 50% methanol/water mixture, and each of the resuspended pellets is mixed with 42 μl of an internal standard solution (IS). A blank containing water/methanol/internal standard without cells is measured in parallel. The internal standard solution is prepared by mixing 200 μl of D3-acetic acid, 200 μl of D6-propionic acid, 200 μl of D8-butyric acid, and 200 μl of 2-ethyl-butyrate (all 1 mM, all from Sigma-Aldrich, Inc.) with 40 μl of 5M NaOH.

The cells are resuspended by stirring the tubes by vortexing, frozen on dry ice for 30 minutes, and thawed at room temperature for 10 minutes. Chloroform (225 μl) is added to the pellet samples before repeating the freeze-thaw cycle of the samples. Similarly, 42 μl of the IS and 225 μl of chloroform are added to 450 μl of the culture medium. The tubes are stirred for 5 seconds by vortexing, and centrifuged at 10,000×g (4° C.) for 5 minutes.

The pH of the upper phases from the sample preparations with various amounts of a 1 mM solution of a short-chain fatty acid standard preparation (consisting of Supelco volatile acid standard mix (Sigma-Aldrich, Inc.)) and the internal standard was adjusted to 11, was dried in a centrifugal evaporator, and derivatized at 80° C. for 60 minutes with 60 μl of a pyridine: MTBSTFA (Soltec Ventures) 1:1 mixture. The samples, the blank, and the standard preparations were analyzed by GC-MS according to the description (Sharma, et al., 2018 PMID 30231992). Metaquant (Bunk, et al., 2006 PMID 17046977) was used to create a standard curve based on the peak area for a specific m/z value of each short-chain fatty acid.

Next, the short-chain fatty acids in the samples are quantified using Metaquant and corrected for the amounts of short-chain fatty acids in the blank value and the recovered amount of the internal standard (as the internal standard, a deuterated standard preparation is used for each short-chain fatty acid, and 2-ethyl-butyrate is used for other short-chain fatty acids).

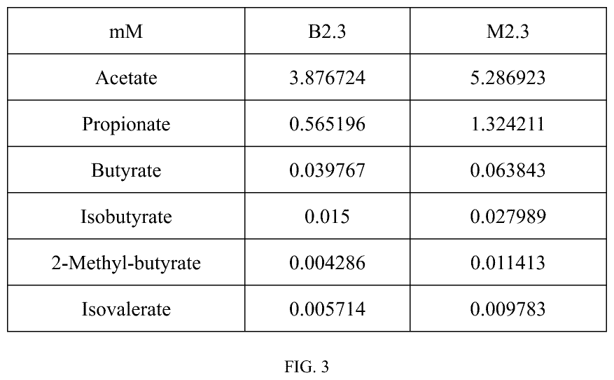

FIG. 3 is a table showing amount of change of short-chain fatty acids according to the above experiment. In this experiment, the group (M2.3) administered with the above-mentioned fermented composition showed an increase in short-chain fatty acids, especially Acetate, Butyrate, Isobutyrate, Isopentanoate, and Propionate, compared to the group administered with brown sugar (B2.3 control).

Next, a fermented composition was prepared by adding mulberries, ginger, and loquat to the present fermented composition and the ingredients of the present fermented composition. “Fermentation procedure” These fermented compositions were tested to see if they could grow Lactobacillus acidophilus, a type of intestinal bacteria known as good bacteria.

For the tests, 6 items were prepared: the present fermented composition, a compressed solution of the present fermented composition, a fermented composition obtained by adding mulberries to the ingredients of the present fermented composition, a fermented composition obtained by adding ginger to the ingredients of the present fermented composition, a fermented composition obtained by adding loquat to the ingredients of the present fermented composition, and a brown sugar solution as a control. The results confirmed that all the 5 types allowed the growth of Lactobacillus acidophilus.

Next, tests were conducted to see whether the production of various short-chain fatty acids, acetate, propionate, butyrate, and isobutyrate increased with the growth of Lactobacillus acidophilus. Six types of solutions as described above was prepared and tests to grow Lactobacillus acidophilus to a certain number in each solution. In these tests, the Lactobacillus acidophilus bacteria grown to 10 7 CUF/ml was prepared, and the productions of various short-chain fatty acids were measured.

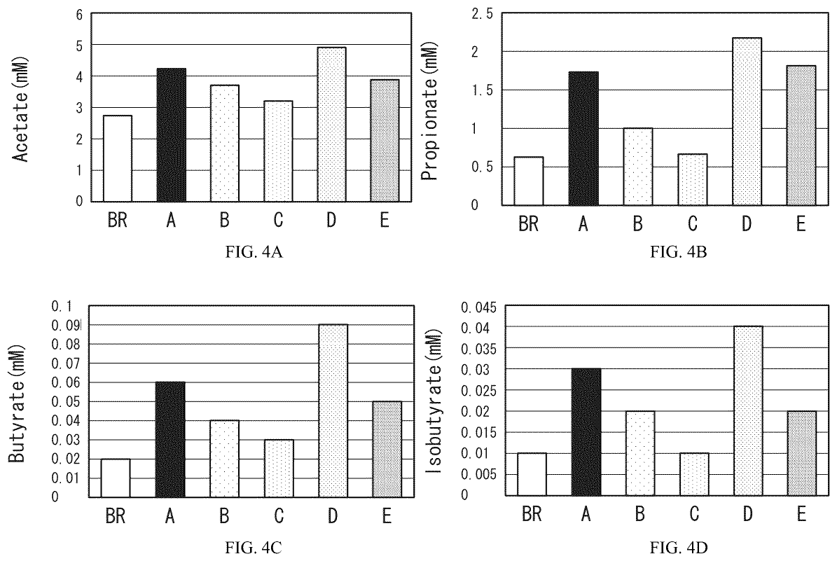

FIGS. 4A-4D are graphs showing concentrations of the various short-chain fatty acids produced by Lactobacillus acidophilus. BR represents the brown sugar in the control only, A represents the present fermented composition, B represents the product obtained by adding loquat to the ingredients of the present fermented composition and performing fermentation, C represents the product obtained by adding ginger to the ingredients of the present fermented composition and performing fermentation, D represents the product obtained by adding mulberries to the ingredients of the present fermented composition and performing fermentation, and E represents the compressed solution of the present fermented composition.

Results in FIGS. 4A-4D showed that all the concentrations of short-chain fatty acids in the fermented composition of the present invention and others, represented high concentration compared to the brown sugar control.

These results showed that the fermented compositions and others act on the Lactobacillus acidophilus to increase the production of above-mentioned short-chain fatty acids. In addition, D (the product obtained by adding mulberries to the ingredients of the present fermented composition and performing fermentation) acts better on acidophilus bacteria compared to the present fermented composition, and increases the production of all the above mentioned short-chain fatty acids. In the meantime, C, fermented by adding ginger to the ingredients of the fermented composition of the present invention acts less on acidophilus bacteria, and decreased the production of all the above mentioned short-chain acids. Thus, the product obtained by the fermentation of the ingredients of the fermented composition of the present invention by adding mulberries acted on the acidophilus bacteria to favorably increase the amount of the production of the above mentioned short-chain fatty acids.

What is claimed is:

1. A prebiotic food product, comprising a fermented composition as an ingredient, wherein the fermented composition is useful to to increase specific short-chain fatty acids in an intestine, and the fermented composition is obtained by fermentation and maturation of one or more kinds of fruits selected from the group consisting of an apple, a persimmon, a banana, a pineapple, an akebia, silver vine, a fig, a wild strawberry, a strawberry, a crimson glory vine, a grape, a mountain peach, a peach, a Japanese apricot, a blueberry, and a raspberry; one or more citrus fruits selected from a navel orange, a hassaku orange, a mandarin orange, a summer tangerine, an orange, an iyokan, a kumquat, a yuzu, a kabosu, a pomelo, a ponkan, a lemon, and a lime; one or more root vegetables selected from a burdock, a carrot, a garlic, a lotus root, and a lily bulb, one or more cereals selected from a brown rice, a glutinous rice, a white rice, a millet, a corn, a wheat, a barley, a foxtail millet, and a Japanese barnyard millet; one or more beans or seeds selected from a soybean, a black bean, a black sesame, a white sesame, an adzuki bean, and a walnut; one or more seaweeds selected from a kelp, a wakame seaweed, a hijiki seaweed, a green laver, and a laver; one or more saccharoses selected from a brown sugar, fructose, and glucose; and one or more selected from honey, starch, a cucumber, a perilla, and a celery, and wherein the fermented composition consists of the following constituents and amino acid composition per 100 g of the fermented composition:
moisture: 5.0 g to 50.0 g,
protein: 0.5 g to 10.0 g,
fat: 0.05 g to 10.00 g,
carbohydrate (sugar): 30.0 g to 75.0 g,
carbohydrate (fiber): 0.1 g to 5.0 g,
ash: 0.5 g to 5.0 g,
β-carotene: 10 μg to 150 μg,
vitamin A effect: 10 IU to 100 IU,
vitamin B1: 0.01 mg to 0.50 mg,
vitamin B2: 0.01 mg to 0.50 mg,
vitamin B6: 0.01 mg to 0.50 mg,
vitamin E: 10.0 mg or less,
niacin: 0.1 mg to 6.0 mg,
calcium: 50 mg to 900 mg,
phosphorus: 200 mg or less,
iron: 1.0 mg to 5.0 mg,
sodium: 20 mg to 300 mg,
potassium: 300 mg to 1000 mg,
magnesium: 40 mg to 200 mg,
sodium chloride equivalent: 0.05 g to 1.00 g, and
copper: 7.0 ppm or less, and
as for the amino acid composition, in 100 g,
isoleucine: 30 to 200 mg,
leucine: 50 to 400 mg,
lysine: 20 to 200 mg,
methionine: 10 to 150 mg,
cystine: 10 to 100 mg,
phenylalanine: 30 to 250 mg,
tyrosine: 20 to 200 mg,
threonine: 40 to 200 mg,
tryptophan: 1 to 100 mg,
valine: 30 to 300 mg,
histidine: 10 to 200 mg,
arginine: 40 to 400 mg,
alanine: 50 to 300 mg,
aspartic acid: 100 to 600 mg,
glutamic acid: 100 to 1200 mg,
glycine: 30 to 300 mg,
proline: 40 to 400 mg, and
serine: 30 to 300 mg.

2. A prebiotic food product, comprising: a fermented composition, the prebiotic food product is obtained by adding one or more selected from the group consisting of a mulberry, a ginger, and a loquat to ingredients of the fermented composition and performing fermentation and maturation, and consists of the above-mentioned composition and amino acid composition.

3. The prebiotic food product according to claim 1, comprising the fermented composition as the ingredient, wherein the fermented composition grows the number of intestinal bacteria, and the intestinal bacteria increase the specific short-chain fatty acid in the intestine.

4. The prebiotic food product according to claim 1, wherein the intestinal bacteria are Bifidobacterium longum.

5. The prebiotic food product according to claim 1, wherein the specific short-chain fatty acid is one or more short-chain fatty acids selected from acetate, butyrate, isobutyrate, isopentanoate, and propionate.

6. The prebiotic food product according to claim 1, comprising the fermented composition as the ingredient and which increases the production of one or more short-chain fatty acids selected from acetate, propionate, butyrate, and isobutyrate.

7. A method for growing the number of intestinal bacteria Bifidobacterium longum in an intestine, wherein the number of intestinal bacteria Bifidobacterium longum increases production of one or more short-chain fatty acids selected from acetate, butyrate, isobutyrate, isopentanoate, and propionate, comprising the step of administering the prebiotic food product according to claim 1 to a subject.

8. A method for growing a number of intestinal bacteria Lactobacillus acidophilus, wherein the number of intestinal bacteria Lactobacillus acidophilus increases production of short-chain fatty acids selected from one or more of acetate, propionate, butyrate, and isobutyrate, comprising the step of administering the prebiotic food product according to claim 1 to a subject.

9. The prebiotic food product according to claim 2, comprising the fermented composition as the ingredient, wherein the fermented composition grows the number of intestinal bacteria, and the intestinal bacteria increase the specific short-chain fatty acid in the intestine.

10. The prebiotic food product according to claim 2, wherein the intestinal bacteria are Bifidobacterium longum.

11. The prebiotic food product according to claim 3, wherein the intestinal bacteria are Bifidobacterium longum.

12. The prebiotic food product according to claim 2, wherein the specific short-chain fatty acid is one or more short-chain fatty acids selected from acetate, butyrate, isobutyrate, isopentanoate, and propionate.

13. The prebiotic food product according to claim 3, wherein the specific short-chain fatty acid is one or more short-chain fatty acids selected from acetate, butyrate, isobutyrate, isopentanoate, and propionate.

14. The prebiotic food product according to claim 4, wherein the specific short-chain fatty acid is one or more short-chain fatty acids selected from acetate, butyrate, isobutyrate, isopentanoate, and propionate.

15. The prebiotic food product according to claim 2, comprising the fermented composition as the ingredient and allows to increase the production of one or more short-chain fatty acids selected from acetate, propionate, butyrate, and isobutyrate.

16. A method for growing the number of intestinal bacteria Bifidobacterium longum in an intestine, wherein the number of intestinal bacteria Bifidobacterium longum increases production of one or more short-chain fatty acids selected from acetate, butyrate, isobutyrate, isopentanoate, and propionate, comprising the step of administering the prebiotic food product according to claim 2 to a subject.

17. A method for growing a number of intestinal bacteria Lactobacillus acidophilus, wherein the number of intestinal bacteria Lactobacillus acidophilus increases production of short-chain fatty acids selected from one or more of acetate, propionate, butyrate, and isobutyrate, comprising the step of administering the prebiotic food product according to claim 2 to a subject.
